(19) United States
(12) Patent Application Publication
(10) Pub. No.: US 2021/0261657 A1
(43) Pub. Date: Aug. 26, 2021

(54) TREATMENT OF CANCER WITH CLEVER-1 INHIBITION IN COMBINATION WITH PD-1/PD-L1 INHIBITOR

(71) Applicant: FARON PHARMACEUTICALS OY, Turku (FI)

(72) Inventors: Maija-Leena HOLLMÉN, Piispanristi (FI); Miro VIITALA, Turku (FI)

(73) Assignee: FARON PHARMACEUTICALS OY, Turku (FI)

(21) Appl. No.: 17/254,795

(22) PCT Filed: Jun. 20, 2019
(86) PCT No.: PCT/FI2019/050482

§ 371 Date: Dec. 21, 2020

(30) Foreign Application Priority Data
Jun. 21, 2018 (FI) 20185563

(51) Int. Cl. C07K 16/28

(52) U.S. Cl. CPC C07K 16/28

(57) ABSTRACT

The invention relates to an agent capable of binding to CLEVER-1 in combination with PD-1 and/or PD-L inhibitor for use in a treatment of cancer.

### FIELD OF THE INVENTION

The present invention relates to an agent capable of binding to CLEVER-1 in combination with PD-1 and/or PD-L1 inhibitor for use in a treatment of cancer.

### BACKGROUND OF THE INVENTION

The huge number of genetic and epigenetic changes that are inherent to most cancer cells provide plenty of tumor-associated antigens that the host immune system can recognize, thereby requiring tumors to develop specific immune resistance mechanisms. An important immune resistance mechanism involves immune-inhibitory pathways, termed immune checkpoints, which normally mediate immune tolerance and mitigate collateral tissue damage. A particularly important immune-checkpoint receptor is cytotoxic T-lymphocyte associated antigen 4 (CTLA4), which downmodulates the amplitude of T cell activation. Antibody blockade of CTLA4 in mouse models of cancer induced antitumor immunity. Some immune-checkpoint receptors, such as programmed cell death protein 1 (PD1), limit T cell effector functions within tissues. By upregulating ligands for PD1, tumor cells block antitumor immune responses in the tumor microenvironment. [1]

Currently, immune checkpoint modulators targeting CTLA-4 and the PD-1/PD-L1 axis are approved for clinical use, and while highly efficacious in about 10-20% of patients with melanoma and certain other tumors, several other important cancer types (such as prostate, breast and colorectal cancer) remain refractory to them [2]. Patients responding favorably to checkpoint inhibition usually have a pre-existing antitumor immune response, which is characterized by high density of IFNγ-producing CD8+ T cells, expression of PD-L1 in tumor-infiltrating immune cells, and high mutational load. Tumors that do not respond to immune checkpoint blockage show either a stromal T cell phenotype where infiltration of T cells (TIL) into tumors or activation of T cells in the tumor microenvironment (TME) is prevented by immunosuppressive stromal compartments, or a non-inflamed phenotype characterized by low T cell infiltration, mutational load and high proliferation of tumor cells. The tumors can be classified immunologically (inflamed non-inflamed) based on the presence of tumor infiltrating cytotoxic CD8 T cells [3]. The inflamed tumors show high mutational load, high IFNγ and PD-L1 expression, and respond favorably to immune checkpoint blocking therapies.

To increase the efficacy of immune checkpoint blockage to refractory tumors scientists have designed combination treatments to convert immunologically ignorant tumors into inflamed type. One such approach is to use PD-1/PD-L1 inhibition with immunogenic cell death inducing chemotherapeutic regimens, such as anthracyclins in order to increase the amount of neoantigens for stimulating long lasting immunity against the tumor [4].

Innate immune cells such as macrophages, however, can dampen T cell activation and contribute to tumor progression despite high mutational load. The macrophages that contribute to tumor-related immunosuppression and provide tumor growth supporting signals may be highly eligible candidates for targeted therapies, since these cells are abundantly present in various tumors, they are very plastic and can be converted into pro-inflammatory macrophages supporting T cell activation and tumor rejection [5, 6]. To date, macrophage targeted strategies under clinical development utilize macrophage colony-stimulating factor receptor inhibition to deplete macrophage populations in tumors [7]. However, resistances to these approaches have already been reported [8]. Thus, there is a need to find novel ways to utilize these cells as an armor to fight against cancer.

In recent years, increasing attention has been paid to the contribution of scavenger receptors in regulating macrophage responses to different stimuli. Clever-1 (also known as Stabilin-1) is a multifunctional molecule conferring scavenging ability on a subset of anti-inflammatory macrophages [9, 10]. In these cells, it is involved in receptor-mediated endocytosis and recycling, intracellular sorting, and transcytosis of altered and normal self-components. More recently, it has been found that the progression of melanoma tumor growth and metastasis is attenuated in Stab1^{-/-} (Clever-1 knock out) mice, and in mice treated with anti-Clever-1 therapy [11].

### SUMMARY OF THE INVENTION

Now, it has been surprisingly found out that Clever-1 inhibition in the tumor microenvironment increases PD-L1 expression on cancer cells due to increased anti-tumor immune responses. This broadens the use of anti-PD-1/PD-L1 therapy in combination with CLEVER-1 inhibition for more effective tumor therapy in non-inflamed unresponsive cancers.

Especially, it has been found that the combination of an agent capable of binding to CLEVER-1 and PD-1 and/or PD-L1 inhibitor(s) is suitable for the treatment of the tumors, which are not responsive to PD-1/PD-L1 inhibitors alone or show only limited responsiveness to PD-1/PD-L1 inhibitors.

Therefore, an object of the present invention to provide a novel treatment for cancer and thus reduce or even eliminate the above-mentioned problems appearing in prior art.

In order to achieve among others the objects presented above, the invention is characterized by what is presented in the characterizing parts of the enclosed independent claims. Some preferred embodiments of the invention will be described in the other claims.

The embodiments and advantages mentioned in this text relate, where applicable, both to the combination of the said agents, the method as well as to the uses according to the invention, even though it is not always specifically mentioned.

The present invention concerns a combination of therapeutically effective amounts of:
(a) an agent capable of binding to Clever-1, and
(b) a PD-1 and/or PD-L1 inhibitor, for use in a treatment of cancer in an individual having diagnosed with a tumor which shows no expression of PD-L1 or shows low expression of PD-L1, and/or which is not responsive to PD-1/PD-L1 inhibitors alone or shows only limited responsiveness to PD-1/PD-L1 inhibitor(s).

Especially, the present invention concerns a combination of therapeutically effective amounts of:
(a) an agent capable of binding to Clever-1, and
(b) a PD-1 and/or PD-L1 inhibitor, for use in treating cancer by reducing malignant tumor growth in an individual and/or by inhibiting metastasis formation.

Now, it has been surprisingly discovered that Clever-1 inhibition increases the expression of PD-L1 in the tumor making it more prone and suitable for anti-PD-1/PD-L1 treatment, and that Clever-1 inhibition promotes the treatment efficacy of PD-1/PD-L1 inhibition in a tumor model that normally is not responsive to PD-1/PD-L1 inhibitor(s) or shows only limited responsiveness to PD-1/PD-L1 inhibitor(s). Hence, the present invention provides a method for increasing PD-L1 expression in patients which are not responsive to PD-1/PD-L1 inhibitor(s) alone or show only limited responsiveness to PD-1/PD-L1 inhibitor(s) and thus improving their response to PD-1 and/or PD-L1 inhibitor treatment. Anti-Clever-1 treatment leads to an increased infiltration of T cells into tumors and in this way increase PD-1/PD-L1 expression for improved targeting with anti-PD-1 and/or anti-PD-L1. Therefore, the present invention provides improved efficacy of anti-PD-1/PD-L1 treatment when combined with Clever-1 targeting to block the negative regulation of T cells in the tumor microenvironment.

The present invention provides a combined treatment of PD-1/PD-L1 inhibitor(s) and anti-Clever-1 agent(s) for cancer patients. According to one aspect, the present invention provides a method for treating or delaying progression of cancer in an individual comprising administering to the individual a therapeutically effective amount of an agent capable of binding Clever-1 in combination with PD-1 and/or PD-L1 inhibitor.

A combination of PD-1/PD-L1 inhibitor(s) and anti-CLEVER-1 agent(s) has been shown to be more effective than PD-1/PD-L1 inhibitors alone in the treatment of cancers, especially in the patients which are not responsive to PD-1/PD-L1 inhibitors alone or show only limited responsiveness for PD-1/PD-L1 inhibitor(s). Especially it has been observed that the combination according to the present invention reduces primary tumor growth and the incidence of lung and lymph nodes metastasis.

### BRIEF DESCRIPTION OF THE DRAWINGS

FIGS. 1A-1C. Clever-1 inhibition increases the expression of PD-L1 on both Louise Lung Cell Carcinoma (LCC) and breast cancer (E0771) models making them more prone to PD-1/PD-L1 inhibition. Stab1^{-/-} are Full Clever-1 knock-out mice and Lyz2-Cre/Stab^{fl/fl} are macrophage specific knock-out mice.

FIG. 2. Immunofluorescence staining of T cells (CD3) in LLC tumors grown in Clever-1 full knockout mice (Stab1^{-/-}), macrophage specific Clever-1 knockout mice (Lyz2-Cre/Stab1^{fl/fl}), or wild type control mice. Figure shows co-staining with Ki67 (cancer cell proliferation) and CD31 (blood vessels).

FIG. 3. The results of T cell activity in Clever-1 deficient LLC tumors. The cytotoxic CD8 T cell infiltration was analyzed with immune checkpoint markers (Lag3⁺ PD-1⁺) by flow cytometry.

FIG. 4. The spread of 4T1 breast cancer cells from the mammary fat pad was monitored by IVIS imaging of lymph nodes and lungs following antibody treatment.

### DETAILED DESCRIPTION OF THE INVENTION

CLEVER-1 is a protein disclosed in the patent publication WO 03/057130, Common Lymphatic Endothelial and Vascular Endothelial Receptor-1. It is a binding protein that mediates adhesion of lymphocytes (and malignant tumor cells) to endothelium in both the systemic vasculature and in the lymphatics. By blocking the interaction of Clever-1 and its lymphocyte substrate, it is possible to simultaneously control lymphocyte recirculation and lymphocyte migration, and related conditions such as inflammation, at the site of lymphocyte influx into, and efflux from, the tissues.

The terms “an agent capable of binding to Clever-1”, “Clever-1 inhibitor” and “anti-clever-1 agent” are interchangeable and refers to agents including antibodies and fragments thereof, peptides or the like, which are capable of binding to Clever-1 for blocking the interaction of Clever-1 and malignant tumor cells. The agent may also be any other inhibitor, such as small molecule inhibitor or macromolecule having an adequate affinity to bind to Clever-1 receptor and to inhibit the protein activity. The term “an antibody or a fragment thereof” is used in the broadest sense to cover an antibody or a fragment thereof which are capable to bind Clever-1 molecule in an individual. Especially, it shall be understood to include chimeric, humanized or primatized antibodies, as well as antibody fragments and single chain antibodies (e.g. Fab, Fv), so long they exhibit the desired biological activities. Particular useful agents are anti-Clever-1 antibodies and fragments thereof. Therefore, according to an embodiment of the present invention the agent capable of binding to Clever-1, i.e. Clever-1 inhibitor or anti-Clever-1 agent, is selected from the group consisting of an antibody or a fragment thereof, peptide(s), macromolecule and any combination thereof.

According to an embodiment of the invention, a method for treating cancer comprises administering to an individual a combination of therapeutically effective amounts of:
(a) an agent capable of binding to Clever-1, such as anti-Clever-1 antibody or fragment thereof, and
(b) a PD-1 and/or PD-L1 inhibitor, such as anti-PD-1/PD-L1 antibody or fragment thereof that binds specifically to a Programmed Death-1 (PD-1) receptor and inhibits PD-1 activity.

The term “treatment” or “treating” shall be understood to include complete curing of a disease or disorder, as well as amelioration or alleviation of said disease or disorder.

The term “therapeutically effective amount” is meant to include any amount of an agent according to the present invention that is sufficient to bring about a desired therapeutic result.

In the present invention, PD-1/PD-L1 inhibitors refers to the inhibitors that are capable of blocking the Programmed Death-1 (PD-1)/Programmed Death Ligand-1 (PD-L1) signaling pathway. PD-1 and PD-L1 inhibitors act to inhibit the association of the programmed death-ligand 1 (PD-L1) with its receptor, programmed cell death protein 1 (PD-1). Upon ligand binding, PD-1 signaling inhibits T-cell activation, leading to reduced proliferation, cytokine production, and T-cell death. In some embodiments, the PD-1/PD-L1 inhibitors are PD-1/PD-L1 binding antagonists, which may comprise antibody or fragments thereof which block the interaction between PD-L1 and its receptor PD-1. The antibody or fragment thereof may bind specifically to PD-1 or to PD-L1 for disrupting the interaction between PD-1 and PD-L1 and inhibiting PD-1/PD-L1 signaling. The anti-PD-1/PD-L1 antibody may be a chimeric, humanized or monoclonal antibody or any fragment thereof. According to the present invention PD-1/PD-L1 inhibitor may be any suitable PD-1/PD-L1 inhibitor and it is selected on the basis of the required treatment. In an exemplary embodiment according to the invention, anti-PD-1/PD-L1 antibody may be selected from nivolumab, pembrolizumab, atezolizumab, avelumab, durvalumab or any combination thereof. These anti-PD-1/PD-L1 antibodies are only examples of the known antibodies used in the field, the present invention is not limited to these.

The present invention for treating cancer by reducing malignant tumor growth and/or by inhibiting metastasis formation is applicable to all forms of cancers. Thus, any benign or malignant tumor or metastasis of malignant tumor can be treated.

Especially, the present invention may be useful for tumors which are not responsive to PD-1/PD-L1 inhibitors alone or show only limited responsiveness to PD-1/PD-L1 inhibitors. When no changes or only slightly changes are observed in tumor growth and/or metastasis, then the patient is classified to be no responsive to PD-1/PD-L1 inhibitors. According to an embodiment of the invention, a combination of PD-1/PD-L1 inhibitor(s) and anti-Clever-1 agent(s) is used in treating an individual having diagnosed with a tumor which shows no expression of PD-L1 or shows only low expression of PD-L1 in the tumor microenvironment. According to an embodiment of the present invention, in addition to no expression or low expression of PD-L1, a tumor may show high macrophage infiltration, low mutational burden and/or low T cell infiltration. According to an embodiment of the invention, a combination of PD-1/PD-L1 inhibitor(s) and anti-Clever-1 agent(s) is used in treating an individual to increase T cell infiltration and PD-L1 expression in tumor-infiltrating immune cells.

Commonly, PD-L1 expression on tumor cells is used to predict patient eligibility for treatment with PD-1/PD-L1 inhibitors. A level of PD-L1 expression may vary based on the type of cells in which PD-L1 is assessed (tumor versus immune cells), or the source and timing for sample collection. The PD-L1 expression in 50% of the viable tumor cells and/or tumor infiltrating lymphocytes present in the sample is typically considered as high expression for PD-L1 and it predicts responsiveness to PD-1/PD-L1 inhibitors. The PD-L1 expression in less than 1% of the viable tumor cells and/or tumor infiltrating lymphocytes present in the sample is considered as no expression, and PD-L1 expression in the range of 1-49% of the viable tumor cells and/or tumor infiltrating lymphocytes present in the sample is also considered as PD-L1 positive but the responsiveness might be dependent on the therapy.

The present invention is based on the finding that Clever-1 inhibition increases PD-L1 expression in tumor cells, and therefore the present invention is not limited to any certain threshold level of PD-L1-positivity, but the present invention is applicable for improving PD-L1 expression independent on the native level. The present invention is most valuable for patients having diagnosed with a tumor which shows no expression or low expression of PD-L1, since then the inhibition of Clever-1 can convert cold tumors hot and increase the efficacy of immunotherapy in patients, which would not normally show treatment response. In the present invention PD-L1 expression in less than 1% of the viable tumor cells and/or tumor infiltrating lymphocytes present in the sample is considered as no expression, and PD-L1 expression in 1-50% of the viable tumor cells and/or tumor infiltrating lymphocytes present in the sample is considered as low expression. Low expression covers also situation with no expression in any cells. Hence, according to an embodiment of the present invention tumors showing low PD-L1 expression comprises tumors showing PD-L1 expression in less than 1% and/or 1-50% or 1-49% of the viable tumor cells in the sample. The percentage is calculated from the total amount of viable cells present in the sample. PD-L1 expression can be evaluated e.g. by using of different staining platforms and antibodies known to a skilled person. The evaluation of PD-L1 expression can be performed any suitable method. Several commercial diagnostic assays for determining PD-L1 expression are available and some of them are specific for certain cancer types or intended to use in combination with specific anti-PD-L1 inhibitors. Hence, according to an embodiment of the present invention the predictive biomarker(s) may be used prior to the treatment for determining patient's responsiveness to anti-PD-1/PD-L1 therapy. The patients which typically responded well to anti-PD-1 therapy had PD-L1-positive and T-lymphocyte-rich tumor specimen. According to an embodiment of the present invention, the expression of PD-L1 may be tested by staining tumor cells with anti-PD-L1. PD-L1 protein expression is typically determined by calculating the percentage of viable tumor cells showing partial or complete membrane staining at any intensity from total amount of the of viable cells present in the sample.

According to an embodiment the present invention, an agent capable of binding to Clever-1 is administered to an individual prior to PD-1 and/or PD-L1 inhibitor. According to another embodiment of the invention, an agent capable of binding to Clever-1 is administrated to the individual simultaneously with PD-1 and/or PD-L1 inhibitor, wherein they are admixed as a single composition or administered concurrently. In an embodiment according to the present invention, an agent capable of binding to Clever-1 and PD-1 and/or PD-L1 inhibitor may be administered sequentially, wherein at least part of the anti-Clever-1 agents are administered prior to PD-1/PD-L1 inhibitors. Administering may be performed, for example once, a plurality of times, and/or over one or more extended periods.

In an embodiment of the present invention, the patient may be firstly treated with PD-1/PD-L1 inhibitor(s) alone and after notifying that the desired treatment response has not been achieved, the treatment by administering anti-Clever-1 agent(s) in combination with anti-PD-1/PD-L1 agents is started.

“Administering” refers to the physical introduction of a composition comprising said therapeutic agents to an individual, using any of the various methods and delivery systems known to those skilled in the art. The agents to be used in the present invention may be administered by any means that achieve their intended purpose. For example, administration may be intravenous, intramuscular, intraperitoneal, intra-tumoral, subcutaneous or other parenteral routes of administration, for example by injection. In addition to the pharmacologically active compounds, the pharmaceutical preparations of said agents preferably contain suitable pharmaceutically acceptable carriers comprising excipients and auxiliaries that facilitate processing of the active agents into preparations that can be used pharmaceutically. The dose chosen should be sufficient to reduce malignant tumor growth and/or inhibit metastasis formation.

In treatment methods according to the present invention, also any other anti-cancer agents may be used in addition to PD-1/PD-L1 inhibitors and anti-CLEVER-1 agents.

### EXPERIMENTAL

The following examples are merely illustrative of the principles of the present invention and are not intended to limit the scope of the invention.

Tumor Models

LLC1 (Lewis lung carcinoma cells) were injected (1×10⁵ cells/mouse) subcutaneously in the flank of Clever-1 full knockout mice (Stab1^{-/-}), macrophage specific Clever-1 knockout mice (Lyz2-Cre/Stab1^{fl/fl}), or wild type control mice and tumor growth was monitored every 3-4 days for 15 days. Stab1^{-/-} are Full Clever-1 knock-out mice and Lyz2-Cre/Stab1^{fl/fl} are macrophage specific knock-out mice. Tumor size was measured with a digital caliper and calculated using the formula Tᵥₒₗᵤₘₑ=π/6×larger diameter×(smaller diameter)². For the E0771 mammary carcinoma model, the tumors were injected into the inguinal mammary fat pad of the genetically modified Clever-1 mice. At the day of sacrifice, the tumors were removed and minced into 1-2 mm³ pieces and thereafter digested with 10 mg/mL collagenase IV (Gibco), 20 μg/mL DNase (Invitrogen), 2.25 μM CaCl₂) in PBS in a shaker heated to 37° C. After 30 min, the tissue was filtered through a 40 μm cell strainer and flushed with 2 mL of MACS buffer (0.5% FBS, 2 mM EDTA in PBS). The resulting single cell suspension was stained with anti-CD45 and anti-PD-L1 and analyzed with LSR Fortessa (BD) for PD-L1 expression on cancer cells. Data were plotted with FlowJo software version 10.4.2. The cancer cells were identified according to their CD45 negativity and size (FSC).

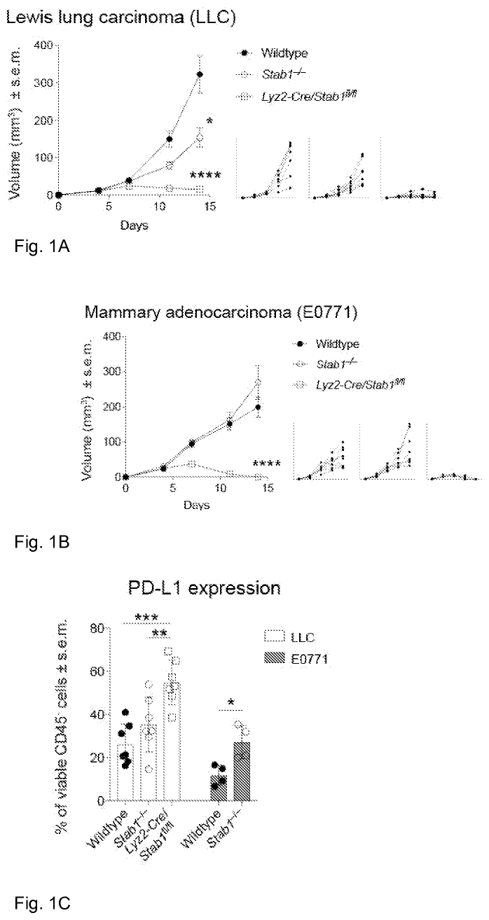

The results are showed in FIGS. 1A-1C. Clever-1 inhibition increases the expression of PD-L1 on both Louise Lung Cell Carcinoma (LCC) and breast cancer (E0771) models making them more prone and suitable for anti-PD-1/PD-L1 treatment.

TIL Analysis from LLC1 Tumors

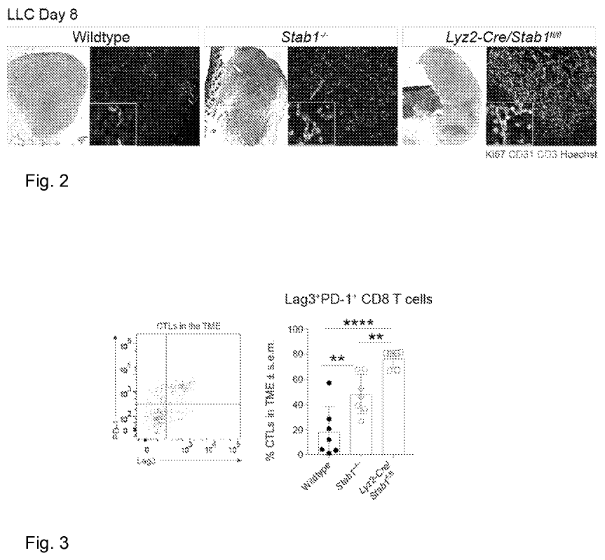

Patients responding to immune checkpoint targeting therapies usually have a pre-existing anti-tumor immune response, which is seen by high infiltration of cytotoxic CD8 T cells and PD-L1 expression in the tumor tissue. For proving whether Clever-1 deletion was sufficient to increase the infiltration of T cells into tumors and this way increase PD-L1 and PD-1 expression for improved targeting with anti-PD-1 or anti-PD-L1, the LLC tumor tissues were stained with anti-CD3 and detected very high infiltration of T cells in the tissue. The immunofluorosence staining of T cells is presented in FIG. 2. Clever-1 full knockout mice (Stab1^{-/-}) and macrophage specific Clever-1 knockout mice (Lyz2-Cre/Stab1^{fl/fl}) shows increased infiltration of T cells compared to wild type control mice. This finding proposes that deletion of Clever-1 can convert cold tumors hot and increase the efficacy of immunotherapy in patients, which would not normally show a response because of low anti-tumor immune responses.

Further, the increased T cell activity in Clever-1 deficient LLC tumors was validated by analyzing cytotoxic CD8 T cell infiltration with immune checkpoint markers (Lag3⁺ PD-1⁺) by flow cytometry. The results are shown in FIG. 3. The CD8 T cells in Clever-1 full knockout mice (Stab1^{-/-}) and macrophage specific Clever-1 knockout mice (Lyz2-Cre/Stab1^{fl/fl}) showed upregulation of both Lag3 and PD-1 indicating an activation of anti-tumor immune responses. This observation proposes improved efficacy of anti-PD-1 treatment when combined with Clever-1 targeting to block the negative regulation of T cells in the tumor microenvironment.

Antibody treatments 1×10⁵ luciferase-expressing 4T1 (Caliper Life Sciences) were injected into the inguinal mammary fat pad of 8-10-week old female Balb/c mice (Charles River). Antibody treatment (i.p.) was started at day 3 after tumor inoculation and was given every 3-4 days until the end of the study with the following doses: anti-Clever-1, 5 mg/kg (clone mStab1, InVivo Biotech); anti-PD-1, 5 mg/kg (clone RMP1-14, BioXCell) as monotherapy or in combination. Combination of mouse IgG1 and rat IgG2a antibodies were used as isotype controls at 5 mg/kg (both from BioXCell). At the day of sacrifice, the mice were injected with 150 mg/kg of D-luciferin substrate (Caliper Life Sciences) and sacrificed after 5 min with CO₂. The tumors, lungs, and lymph nodes were excised and imaged with IVIS after 10 min with the following settings: exposure time=1 second (tumor), 10 seconds (lungs), 30 seconds (lymph nodes), f/stop=1, medium binning, field of view=3.9×3.9 cm². Living Image software was used to quantify the bioluminescent signal reported as units of tissue radiance (photons/s/cm²/sr).

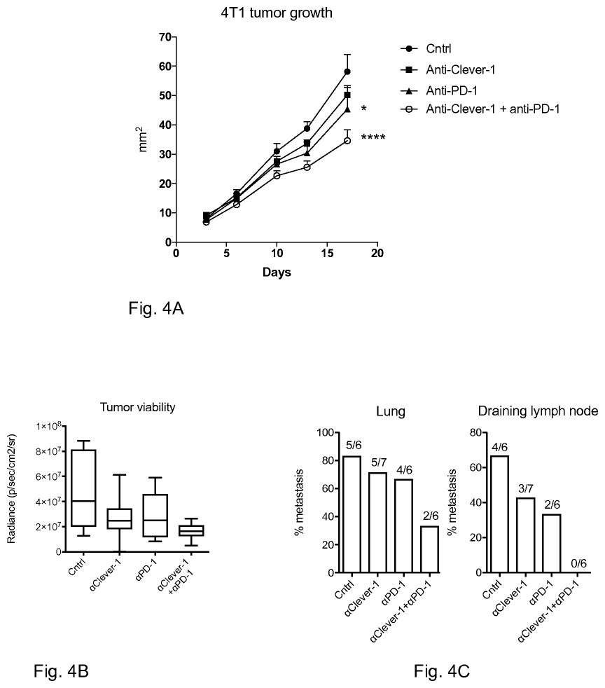

The results are shown in FIGS. 4A-4C. The numbers above each bar indicate how many mice had luciferase-positive metastatic cells relative to the total number of mice in each treatment group. Combination therapy (anti-Clever-1 (mStab) and anti-PD-1) reduced the number of cancer cells in the primary tumor. Anti-Clever-1 in combination with anti-PD-1 stops the spread of 4T1 breast cancer cells to the sentinel lymph nodes. In summary, combined treatment with anti-Clever-1 and PD-1 inhibitor reduces primary tumor growth and the incidence of lung and lymph nodes metastasis.

### CITED REFERENCES

[1] Pardoll D M., The blockade of immune checkpoints in cancer immunotherapy, Nature reviews cancer, 2012, 12(4): 252-264)
[2] Chen D S, Mellman I. Elements of cancer immunity and the cancer-immune set point. Nature 2017; 541(7637): 321-30.
[3] Hegde P S, Karanikas V, Evers S. The Where, the When, and the How of Immune Monitoring for Cancer Immunotherapies in the Era of Checkpoint Inhibition. Clin Cancer Res 2016; 22(8):1865-74 doi 10.1158/1078-0432.CCR-15-1507.
[4] Obeid M, Tesniere A, Ghiringhelli F, Fimia G M, Apetoh L, Perfettini J L, et. al. Calreticulin exposure dictates the immunogenicity of cancer cell death. Nat Med 2007; 13(1):54-61.
[5] Guerriero J L, Sotayo A, Ponichtera H E, Castrillon J A, Pourzia A L, Schad S, et al. Class Ila HDAC inhibition reduces breast tumours and metastases through anti-tumour macrophages. Nature 2017; 543(7645):428-32.
[6] Qian B Z, Pollard J W. Macrophage diversity enhances tumor progression and metastasis. Cell 2010; 141(1):39-51.
[7] Ries C H, Cannarile M A, Hoves S, Benz J, Wartha K, Runza V, et al. Targeting tumor-associated macrophages with anti-CSF-1R antibody reveals a strategy for cancer therapy. Cancer Cell 2014; 25(6):846-59.
[8] Quail D F, Bowman R L, Akkari L, Quick M L, Schuhmacher A J, Huse J T, et al. The tumor microenvironment underlies acquired resistance to CSF-1R inhibition in gliomas. Science 2016; 352(6288): aad3018.
[9] Kzhyshkowska J, Gratchev A, Goerdt S. Stabilin-1, a homeostatic scavenger receptor with multiple functions. J Cell Mol Med 2006; 10(3):635-49.
[10] Kzhyshkowska J, Workman G, CardO-Vila M, Arap W, Pasqualini R, Gratchev A, et al. Novel function of alternatively activated macrophages: stabilin-1-mediated clearance of SPARC. J Immunol 2006; 176(10):5825-32.
[11] Karikoski M, Marttila-Ichihara F, Elima K, Rantakari P, Hollmen M, Kelkka T, et al. Clever-1/Stabilin-1 Controls Cancer Growth and Metastasis. Clinical Cancer Research 2014; 20(24):6452-64.

1. A combination of therapeutically effective amounts of:
(a) an agent capable of binding to Clever-1, and
(b) a PD-1 and/or PD-L1 inhibitor,
for use in a treatment of cancer in an individual having been diagnosed with a tumor which shows no expression or low expression of PD-L1 and/or which is not responsive to PD-1/PD-L1 inhibitors alone or show only limited responsiveness to PD-1/PD-L1 inhibitors.

2. The combination for use in a treatment of cancer according to claim 1, wherein the agent capable of binding to Clever-1 is selected from the group consisting of an antibody or a fragment thereof, peptide(s), macromolecule and any combination thereof.

3. The combination for use in a treatment of cancer according to claim 1, wherein the PD-1 and/or PD-L1 inhibitor comprises an antibody or fragment thereof capable of blocking the interaction between PD-L1 and its receptor PD-1.

4. The combination for use in a treatment of cancer according to claim 1, characterized for use in treating cancer by reducing malignant tumor growth in an individual and/or by inhibiting metastasis formation.

5. The combination for use in a treatment of cancer according to claim 1, wherein the agent capable of binding to Clever-1 is administrated to an individual prior to PD-1 and/or PD-L1 inhibitor(s).

6. The combination for use in a treatment of cancer according to claim 1, wherein the agent capable of binding to Clever-1 is administrated to an individual simultaneously with PD-1 and/or PD-L1 inhibitor.

7. The combination for use in a treatment of cancer according to claim 1, wherein the tumor showing PD-L1 expression shows PD-LI expression in less than 1% and/or 1-50% of the viable tumor cells present in the tumor sample.

8. The combination for use in a treatment of cancer according to claim 1, wherein the tumor further shows high macrophage infiltration, low mutational burden and/or low T cell infiltration.

9. The combination for use in a treatment of cancer according to claim 2, wherein the PD-1 and/or PD-L1 inhibitor comprises an antibody or fragment thereof capable of blocking the interaction between PD-L1 and its receptor PD-1.

10. A method of treating cancer in an individual in need thereof comprising treating said individual with a therapeutically effective amount of an agent capable of binding to Clever-1 and a therapeutically effective amount of a PD-1 and/or PD-L1 inhibitor,
wherein said individual in need thereof is an individual having been diagnosed with a tumor which shows no expression or low expression of PD-L1 and/or which is not responsive to PD-1/PD-L1 inhibitors alone or show only limited responsiveness to PD-1/PD-L1 inhibitors.

11. The method of treating cancer in an individual in need thereof according to claim 10, wherein the agent capable of binding to Clever-1 is selected from the group consisting of an antibody or a fragment thereof, peptide(s), macromolecule and any combination thereof.

12. The method of treating cancer in an individual in need thereof according to claim 10, wherein the PD-1 and/or PD-L1 inhibitor comprises an antibody or fragment thereof capable of blocking the interaction between PD-L1 and its receptor PD-1.

13. The method of treating cancer in an individual in need thereof according to claim 11, wherein the PD-1 and/or PD-L1 inhibitor comprises an antibody or fragment thereof capable of blocking the interaction between PD-L1 and its receptor PD-1.

14. The method of treating cancer in an individual in need thereof according to claim 10, wherein the cancer is treated by reducing malignant tumor growth in an individual and/or by inhibiting metastasis formation.

15. The method of treating cancer in an individual in need thereof according to claim 10, wherein the agent capable of binding to Clever-1 is administrated to said individual prior to PD-1 and/or PD-L1 inhibitor(s).

16. The method of treating cancer in an individual in need thereof according to claim 10, wherein the agent capable of binding to Clever-1 is administrated to said individual simultaneously with PD-1 and/or PD-L1 inhibitor.

17. The method of treating cancer in an individual in need thereof according to claim 10, wherein the tumor showing PD-L1 expression shows PD-LI expression in less than 1% and/or 1-50% of the viable tumor cells present in the tumor sample.

18. The method of treating cancer in an individual in need thereof according to claim 10, wherein the tumor further shows high macrophage infiltration, low mutational burden and/or low T cell infiltration.
